(19) United States
(12) Patent Application Publication
(10) Pub. No.: US 2006/0041387 A1
(43) Pub. Date: Feb. 23, 2006

(54) Smart microarray cancer detection system

(76) Inventor: Xiumei Sun, Seattle, WA (US)

Correspondence Address: JERROLD J. LITZINGER, 2134 MADISON ROAD, CINCINNATI, OH, 45208, US

(21) Appl. No.: 11/205,966

(22) Filed: Aug. 17, 2005

Related U.S. Application Data
(60) Provisional application No. 60/602,006, filed on Aug. 17, 2004.

(51) Int. Cl. C12Q 1/68; G06F 19/00

(52) U.S. Cl. 702/19; 435/6

(57) ABSTRACT

A smart microarray detection system for exploring and detecting cancer specific genes. A microarray is constructed using cancer specific genes that can detect, identify, quantify, and discriminate cancers in biological specimens. Data obtained from the microarray is analyzed with questionable signals reanalyzed with real time PCR. This information is then evaluated using software to determine a cancer prognosis.

### CROSS-REFERENCE TO RELATED APPLICATIONS

This application claims benefit from U.S. Provisional Patent Application Ser. No. 60/602,006 filed Aug. 17, 2004, which application is incorporated herein by reference.

### BACKGROUND OF THE INVENTION

1. Field of the Invention

This invention relates generally to cancer detection and, in particular, to a system for use in early diagnosis of cancer using high-throughput microarray technology.

2. Description of the Related Art

As is well known, cancer is one of the major threats to human health. Late discovery of cancer often leads to the death of patients within 1-2 years. Early diagnosis and treatment is still the only path to lengthen patients' lives, and in some types of cancers, for example, esophageal, breast, stomach and colon cancer, early treatment can extend a patient's life by 10 to 20 more years. Thus, early diagnosis is crucial to improving a patient's life expectancy. However, early diagnosis for some types of cancer has proved difficult due to the deficiency of effective single tumor markers to provide sufficient sensitivity; consequently, early diagnosis is still a problem.

The advanced achievements in both the genome project and microarray technology give a promising glimpse for identifying gene markers for early cancer diagnosis. High-throughput microarray technology is a useful tool in genotyping and relative gene expression profiling. However, the high cost of specialized equipment, optics, as well as variable software, put these techniques out of reach for many researchers. Furthermore, the volume of data produced by high density arrays makes the results difficult to interpret. These microarrays are also quite variable due to different input levels of RNA/cDNA, as about 30% of expression signals are lost using regular commercial software. Relatively higher expression levels could be analyzed using certain commercial software (like GArray), but due to the complexity and difficulty of analysis, this is not easily applicable to array researchers. Microarray experimental results routinely require real time PCR verification of expression levels.

The development of molecular biology provides the possibility for detecting the early changes in human cells. Gene polymorphism, methylation, alteration, overexpression, change of suppression, as well as apoptosis of cells, have all been found to be related to carcinogenesis.

### SUMMARY OF THE INVENTION

It is therefore an object of the present invention to provide a highly sensitive, specific and semi-quantitative tumor gene marker microarray chip with high predictive value and low cost.

It is a further object of the present invention to provide a microarray chip having a low background, a user friendly read out, and ease of analysis.

It is another further object of the present invention to provide a DNA chip which displays libraries that can detect, identify, quantify and discriminate cancers in biological specimens.

It is a still further object of the present invention to provide a microarray chip that can be used for early detection, advanced differential diagnosis and prognosis of certain cancers in clinical hospitals and physicians' offices.

These and other objects of the present invention will be more readily apparent from the enclosed description and drawings.

### DETAILED DESCRIPTION OF THE DRAWINGS

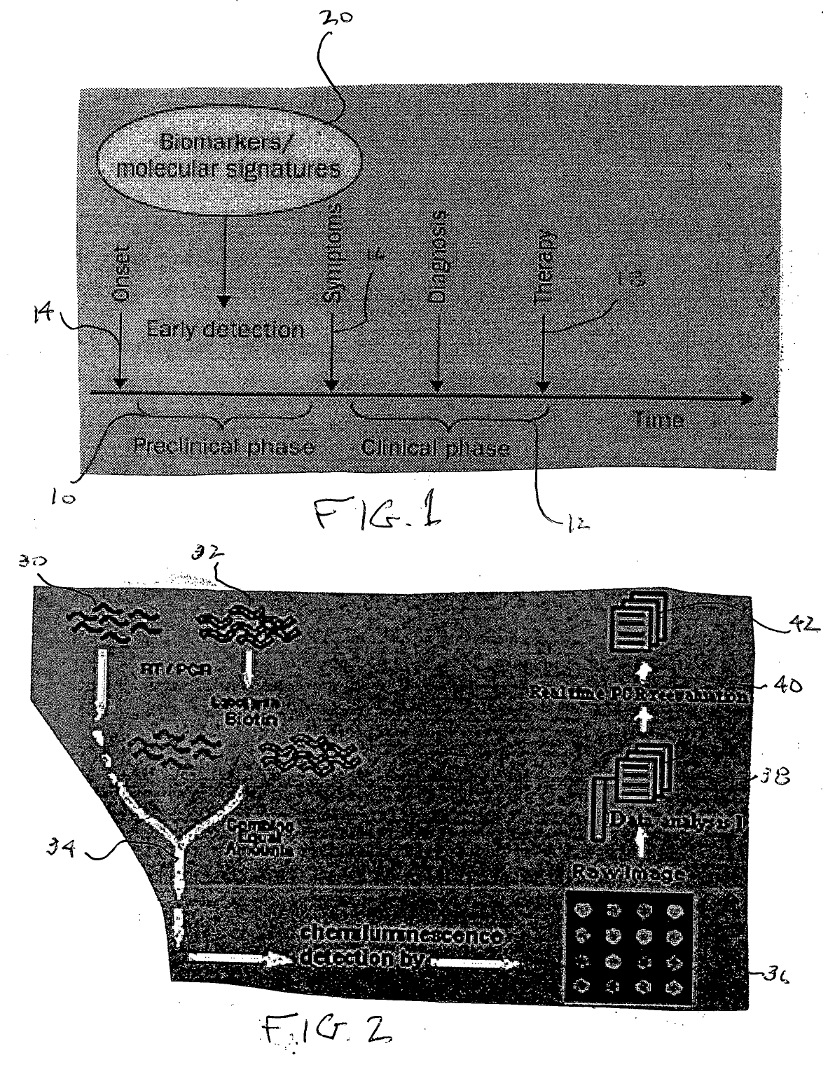

FIG. 1 is a diagram of the progress of cancer versus time; and

FIG. 2 is a flow chart outlining the cancer detection system of the present invention.

### DETAILED DESCRIPTION OF THE PREFERRED EMBODIMENTS

FIG. 1 shows the relationship between biomarkers and cancer progress. Biomarkers are the indicators secreted or expressed in human cells in physiological and pathological conditions. Numerous researchers have found recently that oncogene alteration, polymorphism, methylation, and overexpression of oncoproteins, etc., could result in carcinogenesis independently and, as such, are considered as tumor biomarkers to be used for the early detection of cancers. The formation of cancer is divided into two phases: preclinical stage 10 and clinical stage 12. Preclinical stage 10 starts from the onset 14 of tumor cells to the appearance of visible clinical symptoms 16. Clinical stage 12 starts from the visible clinical symptoms 16 to the end of therapy 18. Early diagnosis can be detected anywhere in preclinical stage 10. Biomarkers 20 are divided into two groups: gene markers and protein markers. With respect to gene biomarkers, some of them reflect the sensitivity of the human body to carcinogenesis (e.g., gene polymorphism); some show the toxicity of genes (e.g., oncogene mutation, deletion, methylation, transcription); some exhibit precarcinoma changes (e.g., oncogene and cell alteration); and some indicate the early formation of cancer cells (e.g., severe oncogene alteration and overexpression).

The development of molecular biology allows the possibility of detecting the early injury or changes in human cells using genetic markers. As mentioned above, gene polymorphism, methylation, alteration, overexpression, change of suppression, as well as apoptosis of the cells, are all found to be related to carcinogenesis.

Esophageal cancer accounts for approximately 1.5% of cancers in the U.S., with an incidence rate approximately 3 times higher in African Americans. In contrast, significantly higher levels occur particularly in men in the Henan province of China and in Iran, Central Asia and parts of South Africa. Certain conditions (e.g., Barrett's esophagus) are linked to increased incidence of esophageal cancer. Esophageal cancers are primarily squamous cell carcinomas or adenocarcinomas, depending somewhat on the location in the esophagus. More than half of the tumors are located in the middle third of the esophagus and approximately 25% in the upper third and approximately 25% in the lower third. Tumors in the upper two-thirds are primarily squamous cell carcinomas, whereas those in the lower third are frequently adenocarcinomas.

While the incidence of stomach cancer has been on the decline, it is still responsible for a significant number of deaths worldwide. Highest incidences are seen in Asia, Eastern Europe and South America. Approximately 90% of gastric cancers are adenocarcinomas. The remaining 10% are typically lymphomas or leiomyosarcomas. Early diagnosis results in improved treatment and increased life expectancy. Lauren's classification describes intestinal (or well differentiated) tumors and diffuse (or undifferentiated) tumors which differ in etiology and pathogenesis. While tumors identified as Stage I have good prognosis and stage IV have poor prognosis, those in stage II and III are comprised of both high and low grade tumors and have different paths of progression. Differential diagnosis would be valuable in defining the type of cancer that would impact treatment regimens.

Helicobacter pylori infection has been closely associated with the development of some gastric cancers. Approximately 60% of gastric tumors are linked to this organism, and strains of Helicobacter pylori with cagA gene are associated with increased pathogenicity. Another Helicobacter hellmannii and Epstein Barr virus have also been implicated in gastric cancer.

In the case of these cancers, numerous studies have been performed to identify potentially overexpressed genes, but all have been done with conventional microarrays to identify genes from many thousands that are differentially expressed. It is an object of the present invention to further identify the best combination of differentially expressed genes that can be used in a user friendly, low cost smart microarray that will be able to differentiate tumor types and eventually provide insights into clinical outcomes.

The overexpression of oncogenes has been detected in many human tumors, including esophageal and stomach cancers. Hundreds of oncogenes have been found related to carcinogenesis. In addition, a number of genes have been demonstrated to be upregulated in esophageal and stomach cancers, and a comprehensive list of potential genes of interest in developing the array are listed in Table 1. Some other traditional markers should also be tested, including CA19-9 and CEA.

TABLE 1

| | | Tumor | |
|---|---|---|---|
| | | suppressor | |
| Cancer Type | Oncogenes | genes | Other |
| Stomach | K-Ras | p53 | Thrombospondin 2 |
| | c-erbB-2 | APC | Matrix Metalloproteinases |
| | | | 1, 7, 10 |
| | Hst-1 | DCC | Keratin 6B |
| | Int-2 | RB-1 | IGF binding protein 3 |
| | Met | E-cadherin | TGF-Beta 3 |
| | myc | | Secreted protein acidic |
| | | | cysteine rich |
| | | | CEA related cell adhesion |
| | | | molecule 6 |
| | | | PDGF receptor beta polypeptide |
| | | | Chondroitin sulfate |
| | | | proteoglycan 2 |
| | | | Cathepsin B |
| | | | Survivin |
| | | | CagA and other HP genes |
| | | | CA-19 |
| | | | DNA methylations eg hMLH1 |
| | | | promoter, Polymorphisms, |
| | | | Apoptosis genes |
| Esophageal | erbB-2 | p53 | Telomerase |
| | myc | p21 | HTERT |
| | CCND1 | p73 (p53 | Egr-1 |
| | | related) H | |
| | FGF3/FGF4 | p16 | c-Fos |
| | EMS1 | | Cyclin D1b |
| | SAS | | PIK3CA |
| | PDFRA | | P160ROCK |
| | BCL2 | | JNK2 |
| | | | RNPEP like protein |
| | | | Human rRNA gene upstream |
| | | | sequence binding |
| | | | transcription factor |
| | | | Uracil DNA glycolase |
| | | | Annexin 2 |
| | | | P300/CBP associated factor |
| | | | Arachidonic acid Metabolism |
| | | | genes |
| | | | Hypoxia Induced Factor 1 alpha |
| | | | DNA methylations eg |
| | | | CDKN2A and MGMT, |
| | | | Polymorphisms, Apoptosis |
| | | | genes |

Previously, gene detection was mainly performed by either PCR or real time PCR, which could detect a single gene one time only, and was not sufficient to distinguish types of cancer. The discovery of microarray technology makes large scale profiling possible. This method enables a parallel analysis of the expression of thousands of genes in one time. With DNA microarrays, transcriptional analysis of more than 10,000 genes can be performed in parallel, enabling the analysis of changes in gene expression upon cellular signaling.

Using DNA microarrays, changes of gene expression between normal and pathophysiological tissue can be compared, and this process has been used for numerous tumor types.

A number of polymorphisms have been associated with esophageal and stomach cancers. Examples of polymorphisms include those in the DNA repair gene XRCC1, GSTP1, Interleukin-1 beta, type II TGF-Beta, BAX and cytochrome P450 2E1.

Increased methylation at CpG islands has become one of the most attractive markers for the development of techniques for the early detection of cancer. Three key features of DNA methylation make it attractive for being tumor markers: (1) CpG islands are rarely methylated in normal tissue but are at high rates in cancers; (2) unlike genetic mutations, which can occur in throughout genes, changes in DNA methylation associated with loss of the transcription of tumor suppressor gene transcription always occur within a specific region of the gene (E-cadherin, p53, p16, and p15 are the best examples), and these have been implicated in esophageal and stomach cancers; (3) DNA methylation changes can be detected in biological fluids such as serum and saliva, making diagnostic test development attractive. Tumor-derived DNA extracted from the serum or saliva of patients has already been successfully used to identify tumor-specific methylation changes in several tumor types; in particular, colon, head and neck, and lung cancer. In the case of esophageal and stomach cancers, hypermethylation has been observed in several genes (e.g., CDK2NA and MGMT in esophageal cancer and in the hMLH1 gene promoter in stomach cancer). In the case of esophageal cancer, these genes are indicated as early predictors of pre-malignant Barrett's, and, in stomach cancer, an indication of microsatellite instability.

Apoptosis is programmed cell suicide and is found to be controlled by many genes. Among these genes, bcl-2 is the one studied most and found to suppress cell apoptosis. Several others have also been implicated in esophageal and stomach cancers, but a comprehensive analysis has not been performed.

Recent studies have found over one hundred oncogenes, tumor suppressor genes, mucins and other proteins which have been observed as up or down regulated or mutated in pancreatic cancers. Some of these genes also provide insight into the susceptibility of the cancer treatments such as radiation or chemotherapy. All these give the promising possibility of developing the appropriate tumor genetic markers using microarray detection system for diagnosis and potentially prognosis of these cancers. Among the genes of interest that will be evaluated in the array are those summarized in Table 2, and are up or down regulated in mucinous and neuroendocrine tumors of the pancreas.

TABLE 2

| Cancer | | |
|---|---|---|
| Type | Genes of Interest | |
| Pancreatic | k-ras | Synuclein gamma |
| | p53□p27 | Metallo protease 2 |
| | maspin | Cyclin E1 |
| | XRCC1 | S100A4 |
| | Her-2/neu | EGF receptor |
| | c-kit | IL-8 |
| | CD59 | TSGF |
| | RhoC | CA242 |
| | TIMP-1 | CA 72.4 |
| | MMP-7 | Beta HCG |
| | Serine/threonine kinase 15 | MUC1, 2, 4 and 6 |
| | c-myc | Neurophilin 1 |
| | Rad51 | Alpha inhibin |
| | Thioredoxin reductase | Neuron specific enolase |
| | CDC28 protein kinase 2 | SLX |
| | caveoline 1 | h-Tert |
| | glypican 1 | ANG-2 |
| | growth arrest-specific 6 protein | NPDC1 |
| | cysteine-rich angiogenic inducer | ELOVL4 |
| | 61 | |
| | Galectin 3 | CALCR |
| | Claudin 4 | Chromogranin A |
| | Cathepsin E | Pancreatic polypeptide |
| | IGFBP3 | Fascin |
| | MBD1 | Prostate stem cell antigen |
| | EDG1 | Cdc/p34 |
| | ADAM9 | Topoisomerase II alpha |
| | NDKA | 14-3-3 sigma |
| | ABL2 | osteopontin |
| | Notch 4 | human polo-like kinase 1 |
| | Heat shock protein 47 | CA19.9 |
| | Ribosomal protein S27a | Villin-1 |
| | KIAA 1705 | p16 |
| | SMAD4 | DCC |
| | FHIT | VEGF |
| | Mesothelin | Stratifin |
| | Kallikrein 10 | Transglutaminase 2 |
| | TMPRSS3 | SOD1 |
| | MKK4 | STK11/LKB1 |
| | DNA hypermethylation genes | Polymorphisms, Apoptosis |
| | | genes |

The conventional DNA microarray technology is regarded to be very expensive and generates a huge amount of data, needing a complex evaluation. However, for several pathological situations, the knowledge of just a limited number of marker genes is suitable for an unequivocal characterization. For instance, using whole genome-wide transcription profiling in leukemias, approximately 50 genes were identified which are characteristic and sufficient for the classification of leukemias. Accordingly, changes in gene expression of selected marker genes can be used for the differential diagnostic of chronic myeloid leukemia and chronic lymphatic leukemia.

In recent genome studies, it has been shown that the knowledge of the expression pattern of 80 genes is sufficient enough to characterize mammary epithelial carcinomas and to give a prognosis for the patients. Over the last 20 years of study, hundreds of oncogenes and tumor suppressor genes have been discovered, and some of them, such as c-erbB-2, have been listed as tumor markers. Some biomarkers have been found to be related to specific tissues; for example, AFP to liver cancer, CA-19 to stomach cancer, CEA to colon and digestive cancer, etc. All these give the basic support for cancer biomarker diagnosis. In addition, use of these different biomarkers in a molecular profile array provide the basis for development of early diagnostic tools.

In DNA microarray studies, genes could be quantified either by imaging colors or real time reading. Thus, the pattern of expression of all the genes in a cell constitutes its gene expression “profile.” Using DNA microarrays, different types of normal and malignant cells can be distinguished from one another because they have distinct gene expression profiles.

An array is an orderly arrangement of samples. It provides a medium matching known and unknown DNA samples based on base-pairing rules and automating the process of identifying the unknown DNA. An array experiment can make use of common assay systems such as microplates or standard blotting membranes, and can be created by hand or make use of robotics to deposit the sample. DNA microarrays provide rapid, cost-effective, and simultaneous screening of genome DNA for numerous sequence variations. Analysis of samples using DNA microarrays is expected to become a standard approach in molecular biology research and clinical diagnostics.

The most common use of this technique is for the determination of patterns of differential gene expression, comparing differences in mRNA expression levels between identical cells subjected to different stimuli or between different cellular phenotypes or developmental stages. Microarray expression analysis has a number of features that have made it the most widely used method for profiling mRNA expression. DNA segments representing the collection of genes to be assayed are amplified by PCR and mechanically spotted at high density on glass microscope slides using relatively simple x-y-z stage robotic systems, thus creating a microarray containing thousands of elements. The microarrays are queried in a co-hybridization assay using two or more fluorescent labeled probes prepared from messenger RNA from the cellular phenotypes of interest. The kinetics of hybridization allows relative expression levels to be determined based on the ratio with which each probe hybridizes to an individual array element. Hybridization is assayed using a confocal laser scanner to measure fluorescence intensities, allowing simultaneous determination of the relative expression levels of all the genes represented in the array.

Efficient expression analysis using microarrays requires the development and successful implementation of a variety of laboratory protocols and strategies for fluorescence intensity normalization. The process of expression analysis can be broadly divided into three stages: (1) array fabrication; (2) probe preparation and hybridization; and (3) data collection, normalization and analysis. Gene expression profiling at the RNA level has been significantly facilitated by microarray analysis and confirmation by quantitative real-time PCR. Microarray analysis offers the advantage of profiling expression levels of hundreds or thousands of genes simultaneously using a single RNA preparation. Although real-time PCR analysis provides precise quantification over a wider dynamic range of expression levels, it is not suited for simultaneous analysis of large numbers of genes. Therefore, array analysis is often used as a tool to screen for target genes that are differentially expressed between biological samples, and real-time PCR provides more accurate quantitative results. Generally, the successful gene expression profiling with microarrays is limited not only by RNA quality and cDNA labeling efficiency, but also by the sensitivity and commercial value. As mentioned above, the expensive confocal digital scanner and the complexity of the re-evaluation testing results by real time PCR highly limits the clinical application of this exceptional technique. However, by using an inexpensive analytic approach to resolve this problem and control the evaluation index in the chip development stage, it could have promising results. Cost effective, highly specific, sensitive, quantitative and complete data, as well as a simple read out, are key components of “smart” microarrays.

The state-of-the-art technology for confirmation and quantitative analysis of expression levels is the highly specific, sensitive quantitative real-time PCR. This methodology has a broader dynamic range of detection (10⁶) than a cy3/cy5 microarray. It is, however, anticipated that with the methods used herein, coupled with real time PCR quality control during chip development, the dynamic range in the smart microarrays will be improved over conventional microarrays, due to lower background and improved signal/noise. Subsequently, algorithms may be generated to enable quantitation by relating to real time PCR expression levels and correcting for microarray bias.

By importing these techniques in the development stage in evaluating smart microarray testing results and establishing quality control methods, cancer detection chips, incorporating the evaluation data with analysis software, can produce semi-quantitative results. All these steps can be developed and quipped into final commercial software and chips; the consumers simply use his software and chips in the above mentioned equipment without re-evaluation result again by real time PCR.

This invention uses reproducible smart DNA microarrays to profile the expression of cancer specific genes and quantitate, as well as validate, microarray results in real time PCR. The target cancers for this embodiment are esophageal, stomach and pancreatic cancers.

There is a great need for the development of microarrays which have improved characteristics over conventional arrays in terms of reproducibility, dynamic range and ease of detection and interpretation. Oncogene product study concerning stomach cancer has shown the diagnostic and prognostic value of c-erbB-2 and ras oncoproteins. The mutation of tumor suppressor gene p53 was also found in stomach cancer. Recent literature studies have found over one hundred oncogenes, tumor suppressor genes and other proteins as up or down regulated or mutated in stomach and esophageal cancers. Some of these genes also provide insight into the susceptibility of the cancer treatments such as radiation or chemotherapy. All these give the promising possibility of developing the appropriate tumor genetic markers using smart microarray detection system for diagnosis, and potentially prognosis, of these cancers. Among the genes of interest that will be evaluated in the smart array for stomach and esophageal cancer are those summarized in Table 1, while genes of interest for pancreatic cancer are summarized in Table 2.

In addition, several genes have been discovered as being upregulated or methylated in these cancers and will be further evaluated in the development of smart microarray chips. Genes related to apoptosis listed below will be evaluated. This includes:
APAF1, ASC, ATM, BAD, BAK, BAX, BCL10, BCL2, BCL2A1, BCL2L1, BCL2L11, BCL2L2, BIK, BIRC1, BIRC1, BIRC2, BIRC3, BIRC4, BIRC5, BIRC6, BLK, BNIP3, BOK, CASP1, CASP10, CASP13, CASP14, CASP2, CASP3, CASP4, CASP5, CASP6, CASP7, CASP8, CASP8AP2, CASP9, CFLAP, CHEK1, CIDEA, CIDEB, CRADD, DAPK2, DFFA, DFFB, FADD, GADD45A, HRK, HUS1, LOC51283, LTA, LTB, LTBR, MCL1, MDM2, MYD88, NOD1, NOL3, P63, RAD53, RIPK1, RIPK2, RPA3, TANK, TNF, TNFRSF10B, TNFRSF10A, TNFRSF10C, TNFRSF10D, TNFRSF12, TNFRSF14, TNFRSF1A, TNFRSF1B, TNFRSF4, TNFRSF5, TNFRSF6, TNFRSF7, TNFRSF8, TNFRSF9, TNFRSF10, TNFRSF11, TNFRSF12, TNFRSF13, TNFRSF14, TNFSF4, TNFSF5, TNFSF6, TRAF5, TRAF6, TRIP, etc.

Hundreds of genes have been identified that are differentially expressed or methylated in the cancers of interest. In addition, studies have identified a number of cancer related genes that are differentially expressed in esophageal and gastric cancer. It is also planned to investigate these genes further, evaluate the significance of these genetic markers, selectively incorporate these genes into a smart user friendly microarray with a user friendly read out, and also further evaluate the accuracy, reproducibility and stability of this methodology. From these studies, it is expected to define the best combination of genetic markers that can be used in defining these cancers, and potentially cancer subtypes, as well as prognosis. It is an objective to develop such smart arrays for a variety of cancers that can be widely used in diagnosis and prognosis.

Arrays of unlabelled genes of interest and implicated in esophageal and gastric cancers will be analyzed along with standards, e.g., β actin, GAPDH and known tumor markers CA19-9 and CEA. These will be hybridized with biotinylated cDNAs derived from both tumor and matched normal tissues. Arrays may be developed using streptavidin labeled with horse radish peroxidase along with luminol, a chemiluminescent substrate. While initially screening larger numbers of genes to design the smart array, it is anticipated using ³²P cDNAs for initial high throughput to quickly determine the subset for the smart array. In either case, microarrays initially will be exposed to X ray film for subsequent scanning analysis with a CCD camera, but a user friendly, cost effective scanning device involving digital imaging could be designed to enable direct detection of luminescence.

DNA microarrays are prepared by spotting microdrops of oligos and proteins of interest at high density onto derivatized glass microscope slides. This technique is widely used, and has been shown to give accurate results. Synthetic oligo arrays are used which have higher accuracy and lower background. RNA will be extracted from normal and matched tumor tissues or biopsies using commercially available RNA isolation methods but emphasizing recent methods that stabilize RNA for extensive periods of time. The test cDNA from normal and tumor samples must be then be labeled before microarray analysis. Conventional microarrays use cy3 or cy5 fluors for labeling and can lead to backgrounds due to residual unincorporated fluors. In the present invention, probes for the microarray are prepared from RNA templates by incorporating biotinylated dC and dU (or ³²P) during 1^{st} strand cDNA synthesis. The purified products are then hybridized to the prepared DNA microarrays comprised of the genes of interest under standard conditions, and captured cRNA targets are quantified using a CCD or digital camera analysis after exposing the normal and tumor microarrays to X ray film. In the case of luminescent detection, chips are treated with horse radish peroxidase labeled streptavidin followed by washing and exposure to luminol. The goal of hybridization is to obtain high specificity of reactivity while minimizing background. It should be noted that, prior to hybridization, the free amine groups on the slide must be blocked or inactivated; otherwise, nonspecific binding of labeled cDNA to the slide can deplete the probe and produce high background. Prehybridization in a solution containing 1% bovine serum albumin may be used to eliminate nonspecific binding of the probe to the slide. Prehybridization and hybridization could be performed under standard protocols.

The preferred system used for early diagnosis of cancer is shown in FIG. 2. Referring now to FIG. 2, a sample 30 of cDNA taken from normal tissue is prepared, along with a sample 32 of cDNA taken from tissue from a tumor. Sample 32 is labeled with biotin, and the cDNA samples are combined in equal amounts and amplified using real time PCR to create a cDNA probe 34. Probe 34 is then hybridized to the prepared DNA microarray comprised of genes of interest for the specific cancer, and the captured cRNA targets are quantified using chemiluminescence and a CCD camera to create a raw image 36. The data obtained from image 36 is analyzed at 38, and then reevaluated at 40 for specific genes using real time PCR. This quantitative analysis is incorporated into smartarray software 42 to create a final diagnosis.

It is anticipated that ˜100 tissue samples of each tumor type (different stages and grade and tumor type and success of treatment) will be initially analyzed during the optimization of the detection procedure and also determining the optimal genes for use in the final smart microarray. In all cases, biotinylated labeled cDNA will be prepared from both tumor and matched normal tissue.

With the selected panel of genes the relationship of the tumor to normal expression seen in the microarray will be compared to that determined by real time PCR to establish a calibration and correct for the bias of the microarray. This will set the groundwork for establishing calibrations that is necessary in the smart array quality control and software development.

The tumor gene marker chips used for cancer diagnosis in the present invention ideally have the following specifications:
i) Highly sensitive, specific and quantitative gene and protein or peptide marker chips;
ii) These gene marker chips have low background, low non-specific oligos, and friendly readout;
iii) These gene marker chips display libraries that can detect, identify, quantify and discriminate cancers and infected diseases in biological specimens and body liquid;
iv) These gene marker chips are composed of different aspects of probes, wherein each aspect of probes selectively interacts with a target associated with a different cause of carcinogenesis, symptoms and tissue specifications;
v) These gene marker chips may include known tumor markers; newly identified tumor markers; genes and oncogenes that are highly related to carcinogenesis; tissue specific markers; peptide and protein segments that are highly related to carcinogenesis and symptoms; oncoproteins that are highly carcinogenesis or causing symptoms, etc;
vi) These gene marker chips may include DNA sequences that can identify pathogens;
vii) These gene marker chips may be composed of different groups of pathogens to identify infected diseases;
viii) These chips may contain 30-400 genes, peptide segments and proteins;
ix) These chips may use nitrocellous membrane, glass or plastic material as plates.

The general process for carrying out the present inventions includes three steps:

I. Array Fabrication

II. Probe Preparation and Hybridization

III. Data Collection, Normalization and Analysis

To fabricate the microarray, careful selection of the genes of interest is required. For example, when diagnosing pancreatic cancer, the genes of interest may be selected from Table 2. The selection of genes involves exploring and optimizing the target genes, and evaluating these genes until about 30-400 are selected. These gene oligos are synthesized using 60-mer 3′ biased oligo for each gene of interest. This can be done by amplification and purification of these oligos, then using an arrayer to spot PCR products onto nylon or nitrocellulose membranes, or glass or plastic slides. This process requires optimizing and choosing the best slide type. Alternatively, these gene oligos could be synthesized directly onto slides which are more specific to avoid impurity and contamination from amplification.

Probe preparation and hybridization is accomplished by isolating total RNA, performing real time PCR and preparing biotinylated tumor cDNA probes for hybridization. Hybridization of the biotinylated probes is carried out using standard protocols.

The third step includes chemiluminescent detection analysis of the microarray, and the data generated by this detection is analyzed by using a commercial software package, such as ScanAlyze or SuperArray. However, this analysis often includes negative, weak positive, unclear or false signals. Therefore, it is necessary to retest these samples with real time PCR. This data about gene expression levels will be expected to be higher than those produced from commercial software due to the high sensitivity and accuracy of real time PCR, which is regarded to be representative of the actual expression patterns.

Repetitive testing from 100-300 different samples until these results are stabilized is then done such that new data will be produced in a numerated way, and the copy numbers of genes expressed in different tumors will be produced and numerated. This quantitative analysis will be incorporated into the new SmartArray software.

Finally, check the genes/protein expression levels with these patients' clinical records, and analyze the relationship between gene/protein levels and patients' diagnosis and prognosis statuses. This data can then be incorporated into the SmartArray software for clinical evaluation.

The advantages of this format of oligo array with chemiluminescent read out is: (a) accuracy; (b) ease of use; and (c) built-in optimization controls for labeling, hybridization and detection which are included in each array. In addition, they are cost effective, with only a thermal cycler, hybridization oven, CCD camera, and compute as required equipment. In addition, they are sensitive and robust, requiring as little as 100 ng of total RNA. As this is very cost effective, the end cost to the patient is minimized.

The technique of the present invention can not only apply to esophageal, gastric and pancreatic cancer, but is also applicable to colon, liver, breast, prostate brain, bone marrow, cardiovascular, blood, gastroenterological, and respiratory tumors. In addition, it could be used to detect infectious diseases (such as HIV and Hepatitis A, B, C, D, E, F, G), sexually transmitted diseases, inflammatory diseases such as respiratory infections, fungi infections, oxygen resistant bacteria infections, and chronic fevers.

What is claimed is:

1) A method for diagnosing cancer in a subject, comprising the steps of:
obtaining a tissue sample taken from a tumor suspected of being cancerous;
obtaining a tissue sample taken from known normal tissue;
labeling both tissue samples using biotin;
performing real time PCR and preparing biotinylated cDNA probes for hybridization;
hybridizating the biotinylated probes using standard protocols;
analyzing the probes using a microarray;
detecting the results using chemiluminescence;
analyzing the date using a commercial software package;
and generating a prognosis of cancer.

2) The method of claim 1, wherein said suspected cancerous tissue is a pancreatic tissue sample.

3) The method of claim 1, wherein said suspected cancerous tissue is a gastric tissue sample.

4) The method of claim 1, wherein said suspected cancerous tissue is an esophageal tissue sample.

5) The method of claim 1, wherein the detecting step further includes scanning the microarray with a CCD image analyzer.

6) The method of claim 1, further including the step of reevaluating the analyzed data by performing real time PCR on the data.

7) The method of claim 1, further including the step of evaluating the reevaluated analyzed data using additional software.

8) The method of claim 1, wherein the commercial software package used is ScanAlyze.

9) The method of claim 1, wherein the commercial software package used is SuperArray.

10) A system for diagnosing cancer in a subject, comprising:
a first tissue sample taken from a tumor suspected of being cancerous;
a second tissue sample taken from known normal tissue;
means for labeling said first and second tissue samples using biotin;
means for performing real time PCR and preparing biotinylated cDNA probes for hybridization;
a hybridization oven for hybridizing the biotinylated probes;
a tumor gene marker microarray chip for generating data relative to a cancer diagnosis;
a CCD image analyzer for scanning said microarray chip to detect the generated data;
computer means for analyzing the data generated using a commercial software package;
means for performing real time PCR again to clarify any noisy signals and any weak and unclear signals;
computer means for reanalyzing the signals and providing clinical diagnostic and prognostic evaluation;
and readout means for displaying prognostic information concerning cancer.

11) The system of claim 10, wherein said suspected cancerous tissue is a pancreatic tissue sample.

12) The system of claim 10, wherein said suspected cancerous tissue is a gastric tissue sample.

13) The system of claim 10, wherein said suspected cancerous tissue is an esophageal tissue sample.

14) The system of claim 10, wherein said commercial software package is ScanAlyze.

15) The system of claim 10, wherein said commercial software package is SuperArray.
